(19) United States
(12) Patent Application Publication
(10) Pub. No.: US 2002/0102211 A1
(43) Pub. Date: Aug. 1, 2002

(54) Use of magnetic resonance imaging in diagnosis of membrane fluidity-related disorders

(76) Inventors: Perry F. Renshaw, Arlington, MA (US); Michael L. Rohan, Cambridge, MA (US)

Correspondence Address: J. PETER FASSE, Fish & Richardson P.C., 225 Franklin Street, Boston, MA, 02110-2804, US

(21) Appl. No.: 10/008,901

(22) Filed: Dec. 7, 2001

Related U.S. Application Data
(60) Provisional application No. 60/254,279, filed on Dec. 7, 2000.

(51) Int. Cl. A61K 49/00

(52) U.S. Cl. 424/9.3

(57) ABSTRACT

The invention relates to methods of diagnosing membrane fluidity-related disorders, or predispositions to membrane fluidity-related disorders, in a subject such as a human patient or an animal, e.g., an animal model of a human disorder. In general, the method includes acquiring a first proton relaxation measurement for a selected region of the brain in a magnetic resonance imaging (MRI) procedure; administering to the subject a challenge that alters physical properties or chemical composition of cell membranes in the brain of the subject; acquiring a second proton relaxation measurement for the selected region of the brain in an MRI procedure; and detecting any difference, e.g., an increase or decrease, between the first proton relaxation measurement and the second proton relaxation measurement, wherein a difference indicates a membrane fluidity-related disorder. The invention also includes methods of assessing the effectiveness of treatments or drugs, e.g., drug candidates in an animal model, for such disorders.

### CROSS-REFERENCE TO RELATED APPLICATION

This application claims priority from U.S. Provisional Patent Application No. 60/254,279, filed on Dec. 7, 2000, which is incorporated herein by reference in its entirety.

### TECHNICAL FIELD

The invention relates to magnetic resonance imaging, biochemistry, neurology, and psychiatry.

### BACKGROUND

Magnetic resonance imaging (MRI) is a medical imaging modality in which data are displayed as images that represent planar sections of physical objects such as the human brain. MRI is based on the ability of certain atomic nuclei to absorb and re-emit electromagnetic radiation at certain frequencies, when placed in an external magnetic field. The predominant source of magnetic resonance signals in the human body is hydrogen nuclei, i.e., protons. In the presence of an external magnetic field, the protons align along the axis of the external magnetic field. Excitation occurs when nuclei in a static magnetic field H are rotated by a transverse magnetic field Hl (perpendicular to the main magnetic field) so that a portion of their magnetic moments lie in the plane perpendicular to the main magnetic field. After excitation, protons precess or wobble around that field direction at a definite frequency known as the Larmor frequency, which depends on the type of nuclei containing the proton and on the local total magnetic field. The precessing protons emit electromagnetic radiation at the Larmor frequency, which can be detected by the same coil that produced the excitation. Image information is acquired by applying additional magnetic fields with a known spatial dependence, usually linear gradients, during signal acquisition; in this way the Larmor frequency for each proton can be made to depend on its position.

One method for imaging utilizes a transmit/receive coil to emit a magnetic field at frequency f₀ which is the Larmor frequency of plane P. Subsequently, magnetic gradients are applied in the x and y directions with well defined waveforms Gₓ(t), G_{y}(t). A signal S(t) is detected in a data collection window over the period of time for which the magnetic gradients are applied. The detected signal S(t) can be expressed as a two-dimensional Fourier transform of the magnetic resonance signal S(t)=∫dxdy ρ(x,y)e^{-ik(t)x}e^{-iq(t)y} with k(t)=2πγ∫₀^{t}Gₓ(t′)dt′, q(t)=2πγ∫₀^{t}G_{y}(t′)dt′ where ρ(x,y) is the proton density of the imaging object. The imaging gradient waveforms and data acquisition are generally ordered so that the data is placed in a two-dimensional matrix Sⱼₗ=∫dxdy ρ(x,y)e^{-2πijx/NΔ}e^{-2πily/NΔ}, where j and l are matrix indices, N is the size of each dimension of the matrix, and Δ is the final image pixel size. The final image matrix Iₙₘ is given by the two-dimensional Fourier transform of Sⱼₗ. Other schemes of acquiring sampling which use other data matrix forms and appropriate transforms are possible.

Image intensity is affected by two characteristic relaxation times (T1 and T2) that vary according to tissue type and chemical and physical cellular environment. These relaxation times are related to exponential signal decay during the acquisition sequence, and image intensity and contrast can be accented by adjusting certain delay times during that acquisition. For a typical sequence known as spin-echo, the final image intensity for a given group of protons can be expressed as

I(x,y)=ρ(x,y)(1−e^{-TR/T1})(e^{-TE/T2})

where ρ(x,y) is the proton density, and T1 (spin lattice decay time) and T2 (spin-spin decay time) are constants of the material related to the interactions of water in cells. Typically, T1 ranges from 0.2 to 1.2 seconds, while T2 ranges from 0.05 to 0.15 seconds. By modifying of the repetition and orientation of excitation pulses, an image can be made T1, T2, or proton density dominated. A proton density image shows static blood and fat as white and bone as black. A T1 weighted image shows fat as white, blood as gray, and cerebral spinal fluid as black. T2 weighted images tend to highlight pathology, because pathologic tissue tends to have longer T2 than normal tissue. T2 is often measured as part of a T2 variant, referred to as T2*, which results from the combination of intrinsic T2 mechanisms and other systematic mechanisms. T2* can be used in place of T2 when these mechanisms are appropriately included.

Omega-3 fatty acids (also known as “n-3” fatty acids) are naturally occurring lipids present at high concentrations in certain species of fish (such as menhaden, mackerel, and salmon) and plant oils (such as flax seed oil and borage oil). Omega-3 fatty acids become incorporated into the lipid bilayer of all cell membranes, as complex phospholipids.

The three most common omega-3 fatty acids are docosahexanoic acid (DHA; 22 carbon chain), eicosapentanoic acid (EPA; 20 carbon chain), and α-linolenic acid (18 carbon chain). By definition, omega-3 fatty acids are polyunsaturated. They contain carbon-carbon double bonds that recur at 3-carbon intervals. The double bonds introduce multiple, rigid bends or kinks in the hydrocarbon chain. The bends or kinks interfere with orderly “packing” of hydrocarbon chains in a lipid bilayer. This lowers the melting point of the omega-3 fatty acid relative to the corresponding saturated fatty acid, and causes the omega-3 fatty acid to increase membrane fluidity when incorporated into membranes. Other types of compounds that become incorporated into membranes and interfere with orderly packing of hydrocarbon chains in the lipid bilayer can also increase membrane fluidity.

It has been reported that cell membranes in neuropsychiatric patients with certain diseases or disorders, e.g., bipolar disorder, differ from the cell membranes of healthy individuals without such diseases or disorders. For example, in bipolar patients, increased fluidity in red blood cell membranes has been observed. This has been related to differences in the hydrocarbon regions of red blood cell membranes, in the phospholipid composition of platelets and red blood cell membranes, and in levels of red blood cell ankyrin, a structural protein found in the membranes of various types of cells, including neurons. There is evidence that omega-3 fatty acids are effective mood stabilizers in patients with bipolar disorder.

### SUMMARY

The invention is based on the discovery that changes in in vivo cerebral membrane fluidity in mammalian subjects, e.g., human or animal subjects or animal models, can be detected through measurements of water proton transverse relaxation time (T2) and longitudinal relaxation time (T1), using conventional MRI systems.

Based on this discovery, the invention features methods of diagnosing a membrane fluidity-related disorder, or a predisposition to a membrane fluidity-related disorder, in a mammalian subject, such as a human patient or an animal, e.g., an animal model of a human disorder. In general, the method includes: acquiring a first proton relaxation measurement for a selected region of the brain of the subject in a magnetic resonance imaging (MRI) procedure; administering to the subject a challenge that alters a physical or chemical property of cell membranes in the brain of the subject; acquiring a second proton relaxation measurement for the selected region of the brain in an MRI procedure after the challenge; and detecting any difference between the first proton relaxation measurement and the second proton relaxation measurement, wherein a difference indicates a membrane fluidity-related disorder.

For example, in Alzheimer's disease and bipolar disorder, cell membranes are stiffer. Thus, methods to monitor increases in fluidity (a decrease in T2) are useful for diagnostic and/or treatment monitoring purposes. In particular, a challenge that indicates an increase in membrane fluidity (decrease in T2) indicates a disorder such as Alzheimer's disease or bipolar disorder. A challenge that results in no significant change in membrane fluidity or T2 indicates no disorder.

A disorder is any abnormal condition or disease, whether caused by a genetic defect, pathogen, physical trauma, chemical agent, or some other cause. Examples of membrane fluidity-related disorders include bipolar disorder, alcoholism, Alzheimer's disease, major depression, and schizophrenia.

The challenge can include administering to the patient an effective amount of a compound such as an omega-3 fatty acid, S-adenosylmethionine, a statin, or a cytidine compound, for an effective length of time. Useful omega-3 fatty acids include docosahexanoic acid, eicosapentanoic acid, and linolenic acid. Certain types of fish oil are useful sources of omega-3 fatty acids. In some embodiments of the invention, the effective length of time for administering omega-3 fatty acids, S-adenosylmethionine, a statin, or a cytidine compound is from 3 days to 6 weeks, e.g., from 5 days to 4 weeks.

Some embodiments of the invention include acquiring a third proton relaxation measurement for the selected region of the brain, e.g., a first measurement prior to challenge, a second measurement at about 4 weeks into a challenge period, and a third measurement at about six weeks into the challenge period.

Preferably, the effective amount of the omega-3 fatty acids is an oral dosage of 0.1 gram to 10 grams per day. In some embodiments, it is about 0.5 gram to about 5 grams per day.

The proton relaxation measurement can be a measurement of a T1 value or a T2 value. The MRI preferably includes acquiring multiple images with incrementally increased or decreased echo time (TE) or repetition time (TR), so that T2 or T1 can be calculated for each pixel. Preferably, the MRI comprises acquiring at least 16 images, e.g., 24 or 32 images, using an echo planar, spin echo imaging sequence. This enhances the reproducibility of the proton relaxation measurement, which preferably is within +/−2%. In some embodiments of the invention, the reproducibility is within +/−1%. As used herein, “T2” refers to the result of any transverse relaxation measurement performed with MRI. The T2 measurement can be taken with any suitable T2 or T2* measurement methods.

The invention also features methods of assessing the effectiveness of a neurological or psychiatric treatment, e.g., a drug candidate, in a subject, e.g., a human patient or in an animal model. One such method includes acquiring a first proton relaxation measurement for a selected region of the brain in a magnetic resonance imaging (MRI) procedure; administering to the subject a neurological or psychiatric treatment; acquiring a second proton relaxation measurement for the selected region of the brain in an MRI procedure; and detecting any difference between the first proton relaxation measurement and the second proton relaxation measurement, wherein a difference indicates that the treatment has an effect on the subject. Multiple drug candidates can be screened using this method.

A second method of assessing the effectiveness of a neurological or psychiatric treatment, e.g., a drug candidate, includes acquiring a first, pre-treatment proton relaxation measurement for a selected region of the brain in a magnetic resonance imaging (MRI) procedure; administering to the subject a pre-treatment challenge that alters a physical or chemical property of cell membranes in the brain of the subject; acquiring a second pretreatment proton relaxation measurement for the selected region of the brain in an MRI procedure; detecting any difference between the first pre-treatment proton relaxation measurement and the second pre-treatment proton relaxation measurement, thereby obtaining a pre-treatment challenge result; administering a neurological or psychiatric treatment to the subject; acquiring a first, post-treatment proton relaxation measurement for a selected region of the brain in an MRI procedure; administering to the subject a post-treatment challenge that alters a physical or chemical property of cell membranes in the brain of the subject; acquiring a second post-treatment proton relaxation measurement for the selected region of the brain in an MRI procedure; detecting any difference between the first post-treatment proton relaxation measurement and the second post-treatment proton relaxation measurement, thereby obtaining a post-treatment challenge result; and comparing the pre-treatment challenge result with the post-treatment challenge result, wherein a difference between the pre-treatment challenge result and the post-treatment challenge result indicates that the treatment has an effect on the subject.

In these methods, a treatment, e.g., a drug candidate, that causes an increase in membrane fluidity (decrease in T2) indicates that the treatment will likely have an effect on disorders such as Alzheimer's disease or bipolar disorder. A treatment that results in no significant change in membrane fluidity or T2 would have no effect on a disorder such as Alzheimer's disease or bipolar disorder.

In another aspect, the invention features a method of diagnosing a membrane fluidity-related disorder, or a predisposition to a membrane fluidity-related disorder, in a subject, by acquiring a proton relaxation measurement for a selected region of the brain in a magnetic resonance imaging (MRI) procedure, thereby obtaining a test value; and comparing the test value with a predetermined range of standard values for proton relaxation measurements, wherein a test value outside the predetermined range of standard values is indicative of a membrane fluidity-related disorder, or a predisposition to a membrane fluidity-related disorder. A “predetermined range of standard values” is a range of values that is empirically determined for a particular field strength magnet and subject. For example, this predetermined range of standard values for a human brain at 1.5 T is about 40 to 100 msec.

Unless otherwise defined, all technical and scientific terms used herein have the same meaning as commonly understood by one of ordinary skill in the art to which this invention belongs. In case of conflict, the present application, including definitions, will control. All publications, patents, and other references mentioned herein are incorporated by reference.

Although methods and materials similar or equivalent to those described herein can be used in the practice or testing of the present invention, useful methods and materials are described below. The materials, methods and examples are illustrative only and not intended to be limiting. Other features and advantages of the invention will be apparent from the detailed description and from the claims.

### DESCRIPTION OF DRAWINGS

FIG. 1 is a graph showing mean T2 values for six bipolar subjects subjected to a six week course of treatment with omega-3 fatty acids.

FIG. 2 is a graph showing mean T2 values for 14 bipolar subjects who underwent an eight-week challenge period wherein they received omega-3 fatty acids (from fish oil) at a dosage of 3 grams per day. Mean T2 values are shown for baseline prior to beginning of challenge, four weeks of challenge, and eight weeks of challenge. The decrease in T2 values for treatment (at 4 weeks and 8 weeks) is highly statistically significant (p<0.01). Clinically, 8 of the 14 patients were noted to have mild to marked improvement with the treatment.

FIG. 3 is a graph showing mean T2 values for 12 healthy control (non-bipolar) subjects who received no treatment during a four-week mock challenge period. Mean T2 values are shown for baseline prior to beginning of mock challenge period, and four weeks later. The increase in T2 values at the four-week point is not statistically significant.

### DETAILED DESCRIPTION

The invention provides for reliable biophysical data to be included with conventional mental and behavioral data when diagnosing neuropsychiatric diseases and disorders that involve altered membrane fluidity. Various diseases and disorders fall into this category, including bipolar disorder, alcoholism, Alzheimer's disease, major depression, and schizophrenia. Knowledge of the role or mechanism of the altered membrane fluidity in the disease or disorder may be useful, but such knowledge is not necessary for practicing the invention. In some situations, MRI data according to the invention may indicate merely that a patient has a predisposition to a disease or disorder. In such situations, those of skill in the art will recognize the need to evaluate the MRI data in the context of other patient signs and symptoms. Preferably, in general, MRI data obtained according to the invention are interpreted and evaluated in the context of other patient signs and symptoms. Knowledge of conventional signs and symptoms for diagnosing membrane fluidity-related diseases and disorders is within ordinary skill in the art.

In addition to providing a diagnostic method, the invention provides an objective method of assessing the effectiveness of a neurological or psychiatric treatment for a membrane fluidity-related disease or disorder. If an alteration in membrane fluidity is positively correlated with a disease or disorder, then as the severity or magnitude of the disease or disorder changes, the magnitude of the alteration in membrane fluidity changes accordingly. This relationship holds true regardless of whether the disease or disorder positively correlates with an increase or a decrease in membrane fluidity. This relationship also holds true regardless of whether the alteration in membrane fluidity is a cause or an effect of the disease or disorder. Assessing treatment efficacy by using the present invention offers advantages over conventional methods of assessing treatment efficacy, because the invention supplies objective measurements of physical changes in cerebral cell membranes and/or changes in the biochemical composition of cerebral cell membranes.

Proton relaxation measurement values for a particular region of the brain can be compared with appropriate standard values to assess the membrane fluidity of cerebral membranes directly, i.e., in absolute terms. If appropriate standard values are not available, however, relative membrane fluidity can be assessed on the basis of how membranes respond to challenge with a biochemical agent that tends to increase or decrease membrane fluidity. For example, if a membrane is in a state of high fluidity, the membrane will be relatively unaffected by challenge with omega-3 fatty acids, which tend to increase membrane fluidity. In contrast, if a membrane is in a state of low fluidity, it will be relatively strongly affected by challenge with omega-3 fatty acids. Conversely, if a membrane is in a state of high fluidity, it will respond to challenge with an agent that tends to decrease membrane fluidity.

Any of various agents that affect membrane fluidity can be employed in the challenge step(s) in methods of the invention. For several reasons, omega-3 fatty acids are particularly suited for use in the challenge step(s). First, omega-3 fatty acids are non-toxic, naturally-occurring nutritional substances. They have beneficial side effects, for example, lowering the risk of cardiovascular disease. Moreover, omega-3 fatty acids are potentially therapeutic for some or all of diseases and disorders that can be diagnosed using the invention.

Other agents that can be employed in the challenge step(s) include S-adenosylmethionine (increases membrane fluidity); cholesterol lowering agents, e.g., statins such as Lipitor® (Pfizer, Inc.)(cholesterol decreases membrane fluidity); and cytidine or cytidine analogs (increase membrane lipid synthesis). A specific example of a statin is lipitor, which could be employed at a dosage of, for example, 10 mg/day. When cytidine is employed as a challenge agent, a suitable daily dosage is 100 to 500 mg. It is to be understood, however, that dosages may vary from the exemplary dosages set forth herein, based on knowledge in the art, e.g., according to the judgment of a qualified physician.

The requisite MRI steps in methods of the invention can be performed using conventional, commercially available MRI systems together with protocols, algorithms, and software known in the art. An example of a suitable MRI system is one that includes a 1.5-T SIGNA™ magnetic resonance scanner manufactured by General Electric Medical Systems, Milwaukee, Wis., equipped with a gradient set capable of echo planar imaging, and a standard quadrature head coil for image detection.

Images of particular regions of interest in the brain are acquired by perturbing the magnetic field in the subject and taking readings at particular times. In some embodiments of the invention, MRI images are acquired using a 1.5 T SIGNA™ magnetic resonance scanner manufactured by General Electric Medical Systems, Milwaukee, Wis. equipped with a echoplanar gradient set capable of whole body imaging, and a standard quadrature head coil for image detection. Examinations can include: 1) anatomical imaging, 2) T2 relaxometry, and 3) DSC MRI cerebral blood volume measurement, in that order. The total examination time can be approximately 1.5 hours.

During each examination, three categories of images can be obtained. The first category corresponds to scout images, typically T1 weighted sagittal images, which serve as a guide to determine the region of the brain that is being viewed. The second category of images corresponds to T1 matched images taken through a predetermined number of planes for which maps of T2 are generated. The T1 matched images typically have a relatively high resolution, e.g., on the order of 1 mm×1 mm or better. The third category of images corresponds to a predetermined number of spin echo, echoplanar image sets, with time of echo or echo time (TE) incremented by a predetermined amount in each consecutive image set through the same axial planes. The data from which the images are produced can be stored in a permanent or temporary storage device using known techniques.

In some embodiments, following structural imaging, subjects are implanted with an 18G angiocatheter in the antecubital fossa for saline and contrast administration, and are repositioned in the scanner for T2 relaxometry. The mid sagittal image from the T1-weighted image series is used to prescribe 10 axial brain slices (7 mm thickness, 3 mm skip) for T2 determinations. For T2 relaxation time measurements, 32 spin echo, echoplanar (EPI) image sets, with TE incremented by 4 msec in each consecutive image set (e.g., TE (1)=32 msec, TE (2)=36 msec, . . . TE (32)=156 msec) are collected with the following parameters (TR=10 msec, slice thickness=7 mm with a 3 mm skip, in-plane resolution=3.125 mm×3.125 mm, FOV=200 mm).

Those of skill in the art will appreciate that any number of planes in the range of about 1 to 40 planes can be used, and that any number of MRI image sets with at least two distinct values of TE can be used. For example, spin-echo echoplanar image sets in the range of 16 to 48 sets can be used. As the number of sets of data increases, accuracy of the estimates of T2 will increase accordingly. It should be appreciated that estimates of T2 derived from smaller numbers of echoes will tend to be less accurate. In some embodiments, many images are acquired using only 2 distinct TE values but with several images acquired at each echo time. This is to improve the statistical significance of the result. Also, the time to repeat or repetition time (TR) is selected having a value in the range of about 3-15 seconds, with about 5 seconds being preferred. Preferably, the slice thickness is about 2 mm to about 10 mm, with about 5 mm being preferred. Preferably, a skip of 0 mm to 3 mm is used. The images can have, for example, an in-plane resolution of about 1.5 mm×1.5 mm, with a field of view of about 200 mm.

The above values produce accurate results with the above-mentioned MR scanner and head coil, but other values can be used, especially if different hardware is employed. It should be appreciated that each of the above values and ranges of values are representative and that values other than the values or ranges described above can also produce accurate results. The above values and ranges were found to produce accurate results with the MR system noted above.

Preferably, the TE-stepped images are corrected for translational and rotational in-plane motion during image acquisition. Such correction can be accomplished, for example, by transferring the images to an offline workstation and using an image registration technique or algorithm, such as the Decoupled, Automated Rotational and Translation (DART) image registration technique, or a variation thereof. The DART technique is described in Maas et al., 1997, “Decoupled, Automated Rotational and Translational Registration for Functional MRI Time Series Data; The DART Registration Algorithm,” Magnetic Resonance in Medicine 37:131-139. It is also described in U.S. Pat. No. 5,850,486. Because images in a progressively incremented TE necessarily diminish in overall image intensity, this must be accounted for in most motion correction schemes.

For example, for T2 maps, values of T2 (x,y) and S (TE=0, x,y) can be calculated on a pixel-wise basis, assuming mono-exponential decay, e.g., ln S(n,x,y)=ln S(TE=0,x,y)−TE (n)/T2 (x,y), where (x,y) describes the location of the pixel, n characterizes the echo number from 1 to 32, and S is the image signal intensity. Linear least-squares regression can be used to calculate a single T2 relaxation time measure for each pixel (x,y).

Delineation of regions and analysis of imaging data can be performed on coded images, and for the experiments described below, the analyst was blind to the identity of the subject. ROIs are selected on 2 to 4 slices through the putamen or cerebellar vermis of DSC MRI images using anatomic boundaries observed in T1 weighted images and an atlas of the brain and cerebellum. The putamen slices are chosen such that the first slice allows for the best visualization of the head of the caudate nucleus. Slice selections for the vermis are based on an assessment of the presence of excessive cerebral spinal fluid (reflecting T2 relaxation times>100 ms) to minimize partial volume artifacts, which result in abnormally elevated T2 relaxation times. ROIs can then be mathematically transformed to T2 maps. Regional T2 relaxation times can be calculated from the median value of all designated pixels across all slices, as the median provides a regional estimate less susceptible to contamination by spurious values from cerebrospinal fluid than the mean. Right and left overall putamen T2 values can be averaged within each subject.

So that the invention may be more fully understood, the following examples are provided. It is to be understood that the following examples are provided for illustrative purposes only, and are not to be construed as limiting the scope or content of the invention in any way.

### EXAMPLES

### Example 1

### Study in Subjects with Bipolar Disorder

A study on omega-3 fatty acids in bipolar disorder was conducted at the McLean Hospital, Belmont, Mass. The study included 30 volunteer patients with bipolar disorder. Inclusion criteria were: (a) meet DSM-IV criteria for bipolar disorder, type I; (b) over age 18; and (c) able to give informed consent. Fourteen patients received omega-3 fatty acids in an open-label fashion. Sixteen patients received a placebo.

The omega-3 fatty acids used were in the form of purified unconcentrated fish oil dispensed as 1000 mg capsules (NordicNaturals™). Each 1000 mg capsule contained 300 mg omega-3 fatty acid, i.e., 200 mg eicosapentanoic acid and 100 mg docosahexanoic acid. Omega-3 fatty acid-treated subjects received 15 capsules per day (5 caps TID), for a total omega-3 fatty acid dosage of 3 grams per day, for a duration of 6 weeks.

Baseline MRI data from the brains of the subjects were collected prior to the six-week period during which the subjects received omega-3 fatty acids or placebo, at four weeks and six weeks after commencement of treatment with omega-3 fatty acids or placebo.

MRI data were in the form of a series of images through the brain. For each image plane, magnetic resonance images were acquired with varied acquisition parameters. For T2 measurements, images were acquired with varying echo times (TE), so that T2 could be calculated for each pixel. A total of 32 images were acquired for each subject using an echo planar, spin echo imaging sequence with incrementally increased TE values. Head movement was detected, and appropriate correction was applied to the imaging data. This was done using the Decoupled Automated Rotation and Translational (DART) motion correction algorithm (Maas et al., 1997, Magnetic Resonance in Medicine 37:131-139).

From the series of images, a single T2 map was generated using standard equations (Yurgelun-Todd et al., 1995, Proc. Soc. Magnetic Resonance, 3rd Scientific Meeting, page 1239). To obtain a global measure of T2, values for all brain pixels within a single axial slice passing through the basal ganglia of the brain were averaged. Regions containing cerebrospinal fluid or non-cerebral tissue were excluded. The reliability of measurement of T2 was within approximately +/−2%.

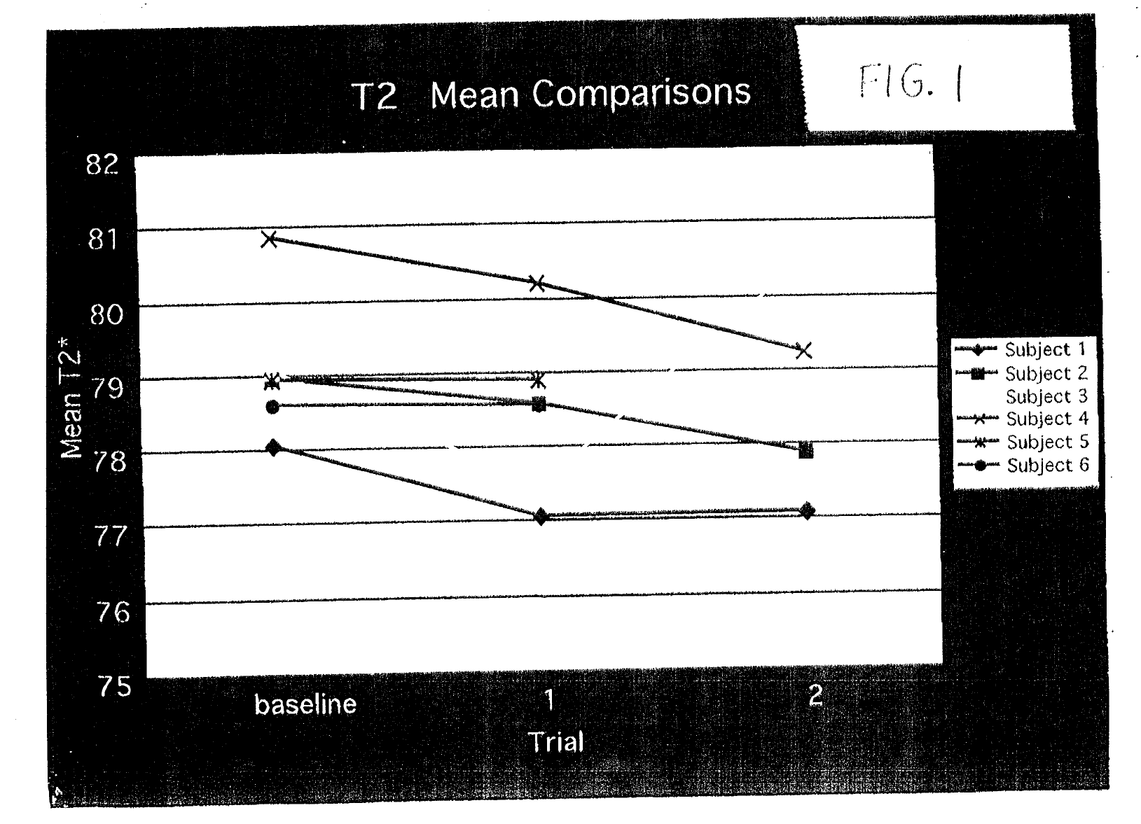

Global estimates of T2 before (baseline) and after treatment with omega-3 fatty acids or placebo were compared for each individual. Representative T2 (mean) comparisons for 6 subjects are shown in FIG. 1. Subjects receiving omega-3 fatty acid demonstrated a significant difference in T2 value. Subjects receiving placebo did not demonstrate any significant change in T2 value. This experiment demonstrates that omega-3 fatty acid supplementation affects brain membranes (which relates to their clinical efficacy), and that T2 relaxation time mapping is useful for establishing diagnoses and following the effects of treatment.

### Example 2

### Study on Bipolar v. Non Bipolar Subjects

A study on omega-3 fatty acids in bipolar disorder was conducted at the McLean Hospital, Belmont, Mass. The study included 14 volunteer patients with bipolar disorder, and 12 normal (non-bipolar subjects). The non-polar subjects received 3 grams of omega-3 fatty acids, as described above in Example 1. The bipolar subjects received the omega-3 fatty acids for 8 weeks.

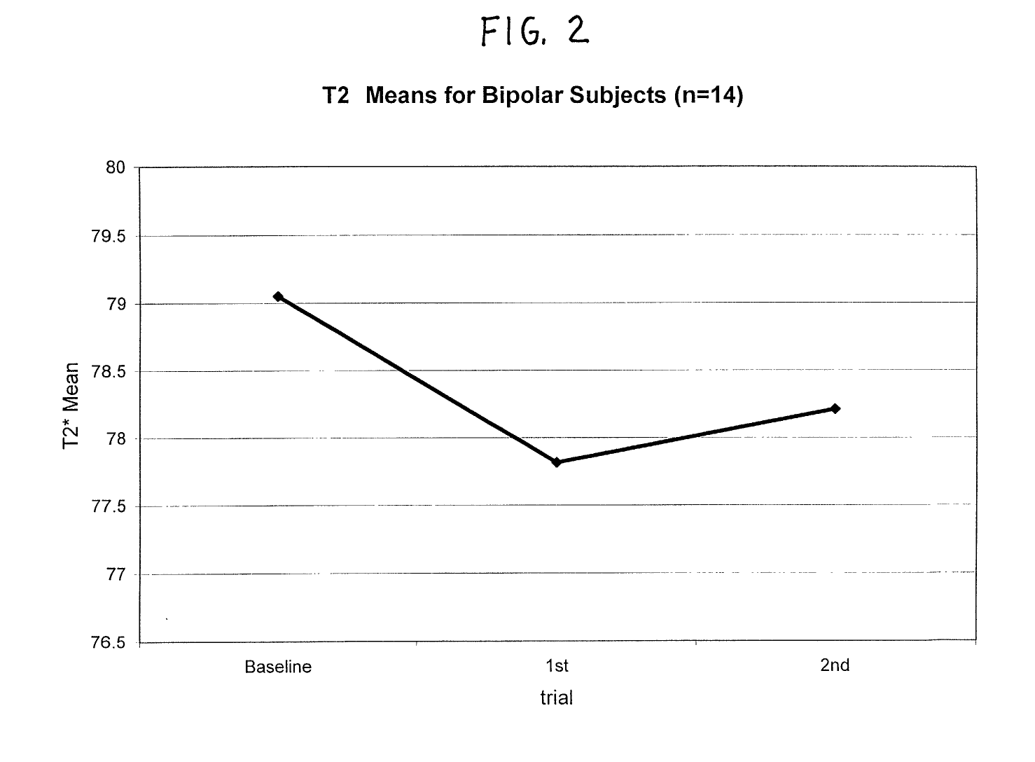

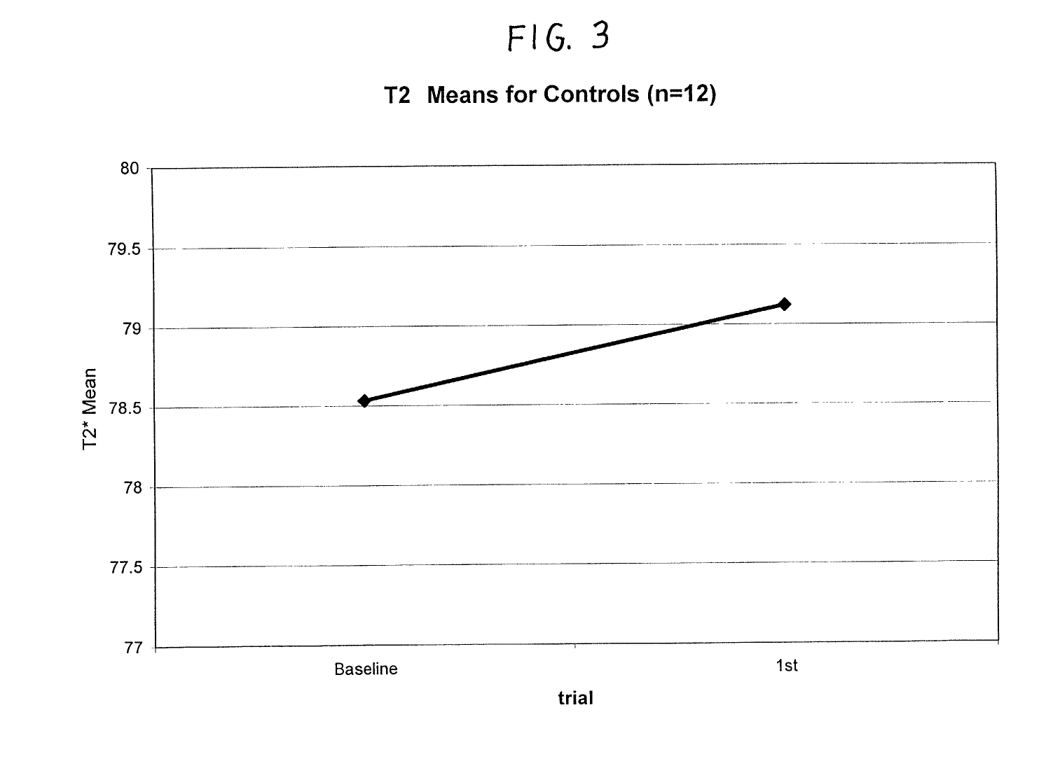

Mean T2 values for the 14 bipolar subjects are shown in FIG. 2, which includes values obtained during a baseline period prior to beginning of challenge (treatment), after four weeks of challenge, and after eight weeks of challenge. The decrease in T2 values for treatment (4 weeks and 8 weeks) was highly statistically significant (p<0.01). Clinically, 8 of the 14 patients were noted to have mild to marked improvement with the treatment. Mean T2 values for the 12 healthy (non-bipolar) subjects are shown in FIG. 3, which includes values for baseline prior to beginning of the mock challenge, and four weeks of mock challenge (no treatment). The increase in T2 values at the four-week point was not statistically significant. MRI was performed as described above in Example 1. This experiment demonstrates that changes in T2 cannot be attributed to placebo effects, and that therefore these changes have clinical significance.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method of diagnosing a membrane fluidity-related disorder, or a predisposition to a membrane fluidity-related disorder, in a mammalian subject, the method comprising:
acquiring a first proton relaxation measurement for a selected region of the brain of the subject in a magnetic resonance imaging (MRI) procedure;
administering to the subject a challenge that alters a physical or chemical property of cell membranes in the brain of the subject;
acquiring a second proton relaxation measurement for the selected region of the brain in an MRI procedure after the challenge; and
detecting any difference between the first proton relaxation measurement and the second proton relaxation measurement, wherein a difference indicates a membrane fluidity-related disorder.

2. The method of claim 1, wherein the disorder is selected from the group consisting of bipolar disorder, alcoholism, Alzheimer's disease, major depression, and schizophrenia.

3. The method of claim 1, wherein the disorder is bipolar disorder.

4. The method of claim 1, wherein a decrease in a T2 proton relaxation measurement after the challenge indicates a disorder.

5. The method of claim 4, wherein the disorder is Alzheimer's disease or bipolar disorder.

6. The method of claim 1, wherein the challenge comprises administering to the subject an effective amount of a compound selected from the group consisting of an omega-3 fatty acid, S-adenosylmethionine, a statin, and a cytidine compound.

7. The method of claim 1, wherein the challenge comprises administering to the subject an effective amount of one or more omega-3 fatty acids for an effective length of time.

8. The method of claim 5, wherein the omega-3 fatty acids comprise a fatty acid selected from the group consisting of docosahexanoic acid, eicosapentanoic acid, and linolenic acid.

9. The method of claim 7, wherein the omega-3 fatty acids are from a fish oil.

10. The method of claim 7, wherein the effective length of time is from 3 days to 6 weeks.

11. The method of claim 7, wherein the effective length of time is from 5 days to 4 weeks.

12. The method of claim 1, further comprising acquiring a third proton relaxation measurement for the selected region of the brain.

13. The method of claim 7, wherein the effective amount of the omega-3 fatty acids is an oral dosage of 0.1 gram to 10 grams per day.

14. The method of claim 7, wherein the effective amount of the omega-3 fatty acid is an oral dosage of 0.5 gram to 5 grams per day.

15. The method of claim 1, wherein the proton relaxation measurement is a T1 value or a T2 value.

16. The method of claim 1, wherein the MRI procedure comprises using incrementally increased or decreased echo times (TE), repetition times (TR), or inversion times (TI).

17. The method of claim 16, wherein T2 is calculated for each pixel.

18. The method of claim 1, wherein the MRI procedure comprises acquiring at least 16 images, using an echo planar, spin echo imaging sequence.

19. The method of claim 1, wherein the reproducibility of the proton relaxation measurement is within 2%.

20. The method of claim 1, wherein the subject is a human.

21. A method of assessing the effectiveness of a neurological or psychiatric treatment in a mammalian subject, the method comprising:
acquiring a first proton relaxation measurement for a selected region of the brain in a magnetic resonance imaging (MRI) procedure;
administering to the subject a neurological or psychiatric treatment;
acquiring a second proton relaxation measurement for the selected region of the brain in an MRI procedure; and
detecting any difference between the first proton relaxation measurement and the second proton relaxation measurement, wherein a difference indicates that the treatment has an effect on the subject.

22. The method of claim 21, wherein the subject is a human patient.

23. The method of claim 21, wherein the subject is an animal.

24. The method of claim 21, wherein a decrease in a T2 measurement indicates that the treatment has an effect on the subject.

25. A method of assessing the effectiveness of a neurological or psychiatric treatment in a subject, the method comprising:
acquiring a first, pre-treatment proton relaxation measurement for a selected region of the brain in a magnetic resonance imaging (MRI) procedure;
administering to the subject a pre-treatment challenge that alters a physical or chemical property of cell membranes in the brain of the subject;
acquiring a second pre-treatment proton relaxation measurement for the selected region of the brain in an MRI procedure;
detecting any difference between the first pre-treatment proton relaxation measurement and the second pre-treatment proton relaxation measurement, thereby obtaining a pre-treatment challenge result;
administering a neurological or psychiatric treatment to the subject;
acquiring a first, post-treatment proton relaxation measurement for a selected region of the brain in an MRI procedure;
administering to the subject a post-treatment challenge that alters a physical or chemical property of cell membranes in the brain of the subject;
acquiring a second post-treatment proton relaxation measurement for the selected region of the brain in an MRI procedure;
detecting any difference between the first post-treatment proton relaxation measurement and the second post-treatment proton relaxation measurement, thereby obtaining a post-treatment challenge result; and
comparing the pre-treatment challenge result with the post-treatment challenge result, wherein a difference between the pre-treatment challenge result and the post-treatment challenge result indicates that the treatment has an effect on the subject.

26. A method of diagnosing a membrane fluidity-related disorder, or a predisposition to a membrane fluidity-related disorder, in a subject, the method comprising:
acquiring a proton relaxation measurement for a selected region of the brain in a magnetic resonance imaging (MRI) procedure, thereby obtaining a test value; and
comparing the test value with a predetermined range of standard values for proton relaxation measurements,
wherein a test value outside the predetermined range of standard values is indicative of a membrane fluidity-related disorder, or a predisposition to a membrane fluidity-related disorder.
